(19) United States
(12) Patent Application Publication
(10) Pub. No.: US 2026/0177484 A1
(43) Pub. Date: Jun. 25, 2026

(54) QUANTITATIVE MOLECULAR DETECTION SYSTEM, IMPROVED STRUCTURE OF QUANTITATIVE MOLECULAR DETECTION CHIP, AND METHOD OF MAKING THE CHIP

(71) Applicant: Dar-Bin SHIEH, Tainan City (TW)

(72) Inventor: Dar-Bin SHIEH, Tainan City (TW)

(21) Appl. No.: 19/125,056

(22) PCT Filed: Dec. 26, 2022
(86) PCT No.: PCT/CN2022/141750

§ 371 Date: Apr. 28, 2025

(51) Int. Cl. G01N 21/64; G01N 33/543

(52) U.S. Cl. CPC G01N 21/6428; G01N 33/54366

(57) ABSTRACT

An improved structure of a quantitative molecular detection chip essentially includes a base portion, a reaction well, and a light-permeable portion. The reaction well is provided in the base portion to receive a to-be-detected substance. The light-permeable portion has a curved surface spaced apart from the reaction well, and the curved surface has an optical focus located in the well space of the reaction well so that light passing through the curved surface can be focused in the reaction well.

### FIELD OF THE INVENTION

The present invention relates to biochip technology and more particularly to a quantitative molecular detection system, an improved structure of a quantitative molecular detection chip, and a method of making the chip.

### DESCRIPTION OF THE RELATED ART

Biochips use micro-electromechanical system (MEMS) technology to induce biological responses or to perform biological analyses such as gene expression analyses, disease diagnoses, drug screening, gene sequencing, and protein analyses. The detection methods of biochips abound, including, for example, optical spectrometry, electrochemical detection, and mass spectrometry. In optical spectrometry, a sample under detection is irradiated with light of a specific wavelength, and the scattered or reflected light resulting from excitation with the light is received and analyzed.

While the prior art uses optical detection, the detection result can be unsatisfactory, and this is mostly because optical detection is relatively sensitive to the light wave signal strength of the scattered or reflected light to be detected. Should the signal strength be slightly lower than required, the accuracy of the detection result will be impaired.

Therefore, further improvement and more efforts in research and development are called for to provide a detection method in which the strength of the light wave signals to be detected can be ensured without having to increase equipment cost, or more particularly without having to raise the light emission intensity of an existing light source.

### SUMMARY OF THE INVENTION

The primary objective of the present invention is to provide an improved structure of a quantitative molecular detection chip, wherein the improved structure can make the light emitted by an external light source converge in a detection area of the detection chip so as to increase the light wave signal strength of the resulting reflected light, thereby overcoming the prior art problem of producing an unsatisfactory detection result.

To achieve the foregoing objective, the improved structure provided by the present invention for a quantitative molecular detection chip essentially includes a base portion, a reaction well, and a light-permeable portion. The reaction well is provided in the base portion to receive a to-be-detected substance. The light-permeable portion has a curved surface spaced apart from the reaction well, and the curved surface has an optical focus located within the well space of the reaction well so that light passing through the curved surface can be focused in the reaction well.

In one embodiment, the well wall and the well bottom of the reaction well are formed with a first hydrophilic surface to prevent the to-be-detected substance from producing air bubbles while the to-be-detected substance is introduced into the reaction well. The air bubbles, if present, may affect the uniformity of the reaction temperature and the accuracy of optical detection or cause leakage of liquid.

In one embodiment, the first hydrophilic surface is formed by a plasma treatment.

In one embodiment, the curved surface of the light-permeable portion is formed by the peripheral contour surface of the base portion.

In one embodiment, the light-permeable portion and the base portion are integrally formed.

In one embodiment, the optical focus of the curved surface is at the center of the reaction well.

In one embodiment, the reaction well is in the shape of a circular tube, and the optical focus of the curved surface is at the center of curvature of the circular tube.

In one embodiment, the base portion has a first side, the well opening of the reaction well is located in the first side, and the present invention further includes a packaging portion provided on the first side of the base portion to close the well opening of the reaction well.

In one embodiment, the packaging portion includes a film layer and a first adhesive layer. The film layer is laid over the first side of the base portion. The first adhesive layer is provided between the film layer and the base portion to bond the film layer to the base portion adhesively.

In one embodiment, the base portion has a plate body, and the plate body has a plate portion, a first side, a second side, an injection hole, an injection channel, an outflow hole, and an outflow channel. The first side and the second side of the plate body are two opposite end faces of the plate portion. The reaction well is provided in the first side of the plate body in a recessed manner. The injection hole is provided in the first side of the plate body in a recessed manner and is spaced apart from the reaction well. The injection channel is provided between the injection hole and the reaction well to bring the injection hole and the reaction well into communication with each other. The outflow hole is far from where the injection hole is located, and the outflow hole is provided in the first side of the plate body in a recessed manner and is spaced apart from the reaction well. The outflow channel is provided between the outflow hole and the reaction well to bring the outflow hole and the reaction well into communication with each other.

In one embodiment, the packaging portion includes a base body, a first channel, and a second channel. The first channel and the second channel correspond in position to the injection hole and the outflow hole respectively and penetrate the base body, and each of the wall surface of the first channel and the wall surface of the second channel is formed with a second hydrophilic surface. When the base body is provided on the first side of the base portion, the first channel and the second channel are aligned with, connected to, and in communication with the injection hole and the outflow hole respectively.

In one embodiment, the second hydrophilic surfaces are formed by a plasma treatment.

In one embodiment, the packaging portion further includes a second adhesive layer provided between the base body and the base portion to bond the base body to the base portion adhesively.

In one embodiment, the packaging portion further includes: a plug removably fitted in the opening of the first channel that is far from the base portion and is formed in an end side of the base body, in order to cut off communication between the first channel and the outside; and a plug removably fitted in the opening of the second channel that is far from the base portion and is formed in the end side of the base body, in order to cut off communication between the second channel and the outside.

In one embodiment, the base portion has a second side facing away from the first side, and the present invention further includes a temperature regulating portion and a sensing portion. The temperature regulating portion is provided on the second side of the base portion and corresponds in position to the reaction well so as to regulate the temperature of the to-be-detected substance received in the reaction well. The sensing portion is electrically connected to the temperature regulating portion and is at a position adjacent to the temperature regulating portion.

In one embodiment, the temperature regulating portion and the sensing portion are provided on a main body and are electrically connected, each through a different feedback circuit, to a processing portion. The processing portion receives the signal detected by the sensing portion and controls the operation of the temperature regulating portion accordingly.

In one embodiment, the temperature regulating portion has a planar heating and cooling loop that matches the reaction well in shape and that is provided on an area of the main body that overlaps with the reaction well.

In one embodiment, the sensing portion has a temperature sensing point provided on the main body at a position corresponding to the reaction well, and the planar heating and cooling loop surrounds the temperature sensing point.

The present invention further provides a quantitative molecular detection system that includes a machine frame, a chip having the foregoing structure, a light source, an image capture portion, and a heat dissipation portion. The chip is provided on the machine frame. The light source is provided on the machine frame, and the light source generates incident light projected to the reaction well of the chip. The image capture portion is provided on the machine frame and receives fluorescence resulting from excitation with the incident light. The heat dissipation portion is provided on the machine frame at a position adjacent to the chip to cool the chip.

In addition, the present invention provides a method of making a quantitative molecular detection chip, and the method includes the following steps:
Step A: obtaining the foregoing base portion;
Step B: performing a plasma treatment on the well wall of the reaction well provided in the base portion, in order to form a first hydrophilic surface;
Step C: attaching an adhesive patch to the base portion, wherein the adhesive patch carries a temperature regulating portion for heating and cooling the to-be-detected substance received in the reaction well; and
Step D: closing the well opening of the reaction well by a packaging technique.

In one embodiment, the packaging technique includes: adhesively attaching one side of a film layer to the base portion such that the film layer is adhesively bonded to the base portion and thereby closes the reaction well, wherein the side of the film layer is formed with a first adhesive layer.

In one embodiment, the packaging technique includes: providing a liquid injection base on the base portion, wherein the liquid injection base has a base body and a channel penetrating the base body, and the channel is in communication with the reaction well when the base body is provided on the base portion; and fitting a plug into the channel to cut off communication between the channel and the outside.

In one embodiment, the method of making a quantitative molecular detection chip further includes performing a plasma treatment on the wall surface of the channel to form a second hydrophilic surface.

In one embodiment, the method of making a quantitative molecular detection chip is such that, by providing the base body on the base portion, a second adhesive layer is provided between the base body and the base portion to bond the base body to the base portion adhesively.

### BRIEF DESCRIPTION OF DRAWINGS

FIG. 1 is an assembled perspective view of the quantitative molecular detection chip provided by the first embodiment of the present invention.

FIG. 2 is an exploded perspective view of the quantitative molecular detection chip provided by the first embodiment of the present invention.

FIG. 3 is a sectional view taken along line 3-3 in FIG. 1.

FIG. 4 is a system block diagram showing a specific way in which the first embodiment of the present invention can be used.

FIG. 5 is an assembled perspective view of the quantitative molecular detection chip provided by the second embodiment of the present invention.

FIG. 6 is an exploded perspective view of the quantitative molecular detection chip provided by the second embodiment of the present invention.

FIG. 7 is a sectional view taken along line 7-7 in FIG. 5.

FIG. 8 schematically shows the quantitative molecular detection system provided by the third embodiment of the present invention.

FIG. 9 is a system block diagram of the quantitative molecular detection system provided by the third embodiment of the present invention.

### DETAILED DESCRIPTION OF THE INVENTION

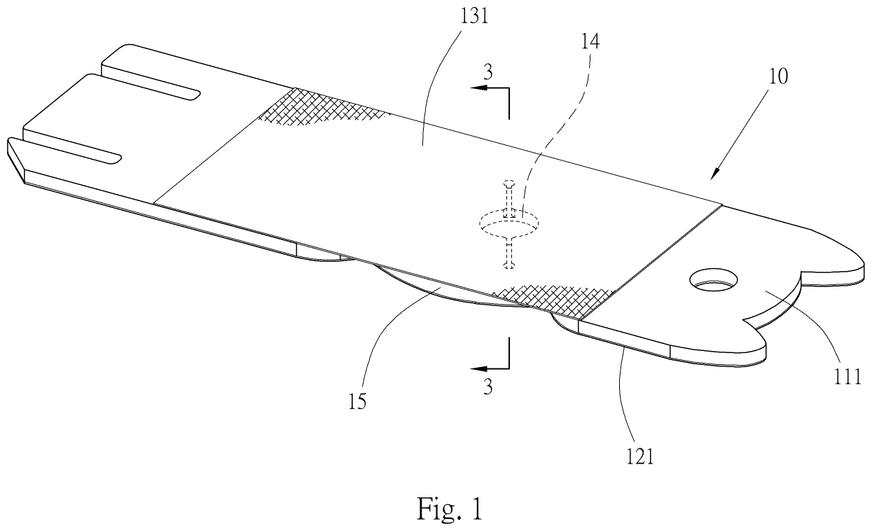

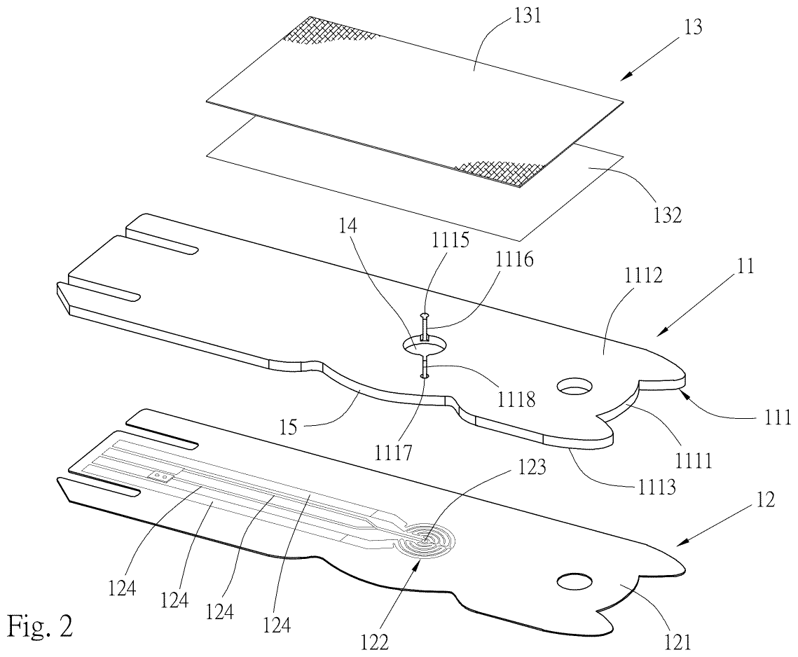

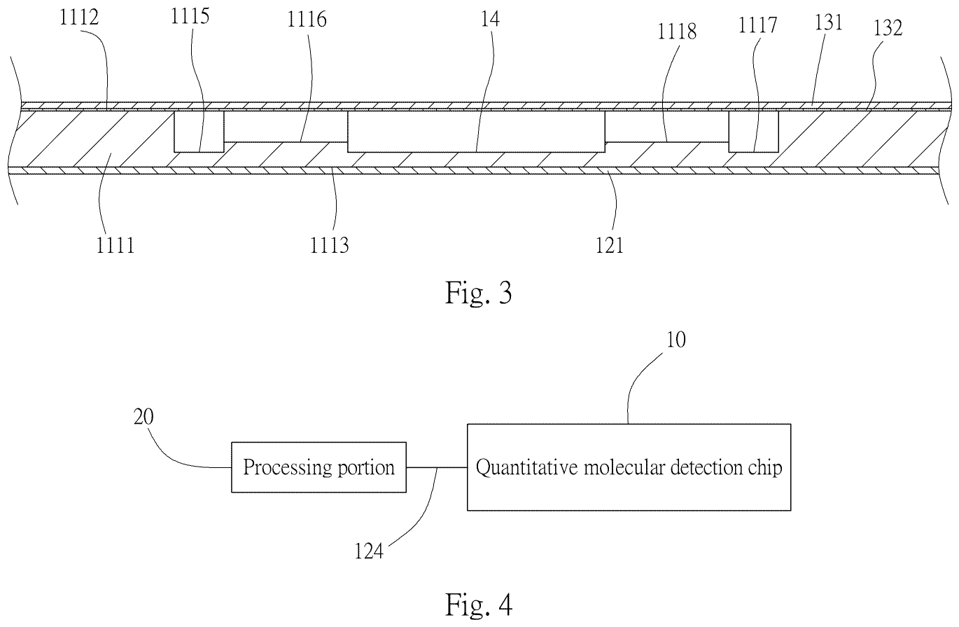

To begin with, please refer to FIG. 1 to FIG. 3 for the quantitative molecular detection chip 10 provided by the first embodiment of the present invention. The quantitative molecular detection chip 10 essentially includes a base portion 11; a sensing, heating, and cooling unit 12; a packaging portion 13; a reaction well 14; and a light-permeable portion 15.

The base portion 11 has a plate body 111, and the plate body 111 has a plate portion 1111, a first side 1112, a second side 1113, an injection hole 1115, an injection channel 1116, an outflow hole 1117, and an outflow channel 1118. The plate portion 1111 is a rigid plastic material such as polycarbonate (PC) and is transparent to allow passage of visible light. The first side 1112 and the second side 1113 are two opposite end faces of the plate portion 1111.

The reaction well 14 is provided in the first side 1112 of the plate body 111 in a recessed manner to receive a to-be-detected substance. More specifically, the reaction well 14 in this embodiment has a circular tube-shaped structure. In other feasible modes of implementation, however, the shape surrounded by the well wall of the reaction well 14 may be any other shape such as a rectangular shape, a diamond shape, or other polygonal shapes.

The injection hole 1115 is provided in the first side 1112 of the plate body 111 in a recessed manner and is spaced apart from the reaction well 14. The injection channel 1116 is provided between the injection hole 1115 and the reaction well 14 to bring the injection hole 1115 and the reaction well 14 into communication with each other. The outflow hole 1117 is provided in the first side 1112 of the plate body 111 in a recessed manner and is spaced apart from the reaction well 14 in such a way that the reaction well 14 is located between the injection hole 1115 and the outflow hole 1117. The outflow channel 1118 is provided between the outflow hole 1117 and the reaction well 14 to bring the outflow hole 1117 and the reaction well 14 into communication with each other.

The sensing, heating, and cooling unit 12 has a main body 121, a temperature regulating portion 122, a sensing portion 123, and feedback circuits 124. The main body 121 is a flexible polyimide (PI) film, matches the peripheral contour shape of the plate body 111 in design, and has a thickness of 0.025 mm. The main body 121 is adhesively attached to the second side 1113 of the plate body 111. The sensing portion 123 has a temperature sensing point, is provided on the side of the main body 121 that faces the plate body 111, and corresponds in position to the reaction well 14. The temperature regulating portion 122 has a planar heating and cooling loop, and the planar heating and cooling loop matches the reaction well 14 in shape, is provided on the side of the main body 121 that faces the plate body 111, and is located on an area of the main body 121 that overlaps with the reaction well 14, with the planar heating and cooling loop surrounding the temperature sensing point. More specifically, the planar heating and cooling loop includes a plurality of concentric circles that are centered at the temperature sensing point and have diameters ranging from 8 mm to 10 mm. The planar heating and cooling loop detects the temperature of the to-be-detected substance in the reaction well 14 by applying the principle of a type-T thermocouple. The feedback circuits 124 are provided on the main body 121 by an existing flexible circuit manufacturing process and form a plurality of pins on one side of the main body 121. The pins allow the temperature regulating portion 122 and the sensing portion 123 to be electrically connected, in a separate manner, to an external processing portion 20 as shown in FIG. 4, in order for the processing portion 20 to receive the temperature signal detected by the sensing portion 123 and control the operation of the temperature regulating portion 122 according to the temperature signal. The material of the feedback circuits 124 may be copper, constantan, indium tin oxide, another metal, electrically conductive carbon, or a combination of the above. In this embodiment, the feedback circuits 124 are made of copper and constantan, and the temperature regulating portion 122 and the sensing portion 123 are integrated by eutectic bonding, which is a conventional technique and therefore will not be detailed herein. In addition, the side of the main body 121 that is provided with the feedback circuits 124, the temperature regulating portion 122, and the sensing portion 123 may be further provided with a PI film by adhesive attachment.

It can be known from the above that the sensing, heating, and cooling unit 12 can be viewed as an adhesive patch having temperature sensing and controlling functions and is adhesively attached to the base portion 11.

The packaging portion 13 is transparent, allows passage of visible light, and includes a film layer 131 and a first adhesive layer 132. The film layer 131 is a polymer film having a thickness of 0.05 mm and is laid over and fixed to the first side 1112 of the plate body 111 to close the reaction well 14, the injection hole 1115, the injection channel 1116, the outflow hole 1117, and the outflow channel 1118 at the same time. The film layer 131, however, allows the to-be-detected substance and a reagent to be input into the reaction well 14 by passing through the film layer 131. The first adhesive layer 132 is a polymer sealing film having a thickness of 0.125 mm, covers the film layer 131 to close the injection hole in the film layer, and provides optical transparency. The technique by which the film layer 131 is fixed to the plate body 111 is not limited to adhesive bonding; other fixing methods such as ultrasonic welding and the use of a sealing adhesive can produce the same effect.

The well wall and well bottom of the reaction well 14, the hole wall of the injection hole 1115, the wall surface of the injection channel 1116, the hole wall of the outflow hole 1117, and the wall surface of the outflow channel 1118 are each formed with a first hydrophilic surface. In other words, any surface of the chip that may be in contact with the to-be-detected substance while the to-be-detected substance flows through, or is stored in, the aforesaid well, holes, and channels is provided with the first hydrophilic surface. The first hydrophilic surfaces are formed by a plasma treatment, with the most preferred embodiment of the plasma being oxygen plasma, and the second most preferred embodiment being argon plasma. During the plasma treatment, the surfaces of the base portion 11 that are subjected to the action of the light and ions of the plasma generate free radicals, and the free radicals are so active that they have chemical reactions with other substances easily and can therefore be used as a medium for bonding substances together. The table below shows the results of a water contact angle test performed on a surface that was not plasma-modified and on first hydrophilic surfaces that were separately modified under a first condition and a second condition. The first condition involved a voltage of 100 W, a fixed oxygen flow rate of 50 sscm, and a test period of 5 minutes. The second condition involved a voltage of 200 W, a fixed oxygen flow rate of 50 sscm, and a test period of 15 minutes. The data in the following table shows that the first hydrophilic surface modified under either condition had significantly higher hydrophilicity than the surface without plasma modification. Therefore, it can be inferred that when the to-be-detected substance flows past the first hydrophilic surfaces, air bubbles are kept from being produced, and this contributes to maintaining the uniformity of the reaction temperature and the accuracy of optical detection or preventing leakage of liquid.

| | Group 1 | Group 2 | Group 3 | Average |
|---|---|---|---|---|
| Surface without plasma modifica- | 86.96 | 85.60 | 83.96 | 85.51 |
| tion | | | | |
| First hydrophilic surface (modified | 47.05 | 47.34 | 43.09 | 45.83 |
| under first condition) | | | | |
| First hydrophilic surface (modified | 5.59 | 5.59 | 10.63 | 7.27 |
| under second condition) | | | | |

The light-permeable portion 15 has a curved surface spaced apart from the reaction well 14. The curved surface is integrally formed by the peripheral contour surface of the plate body 111 in such a way that the curved surface has an optical focus located in the well space of the reaction well 14, allowing light that passes through the curved surface to be focused in the reaction well 14. More specifically, the optical focus of the curved surface is at the center of the reaction well 14 in order for the light to be focused in the reaction well 14, with the light spot of the light covering an area that extends outward from the reaction well 14 for at least 5 mm.

The steps of making the quantitative molecular detection chip 10 having the foregoing structure are as follows:

Step A: The aforesaid base portion 11 is obtained.

Step B: A plasma treatment is performed on the well wall of the reaction well 14 provided in the base portion 11, in order to form a first hydrophilic surface.

Step C: The aforesaid adhesive patch having temperature sensing, heating, and cooling functions is attached to the base portion 11. The adhesive patch having temperature sensing, heating, and cooling functions carries the temperature regulating portion 122 for heating and cooling the to-be-detected substance received in the reaction well 14, wherein the cooling is carried out by air cooling thanks to the high thermal conductivity of the metal loop.

Step D: The well opening of the reaction well 14 is closed by a packaging technique, wherein the packaging technique includes: having the side of the film layer 131 that is formed with the first adhesive layer 132 adhesively attached to the base portion 11, such that the film layer 131 is adhesively bonded to the base portion 11 and closes the reaction well 14.

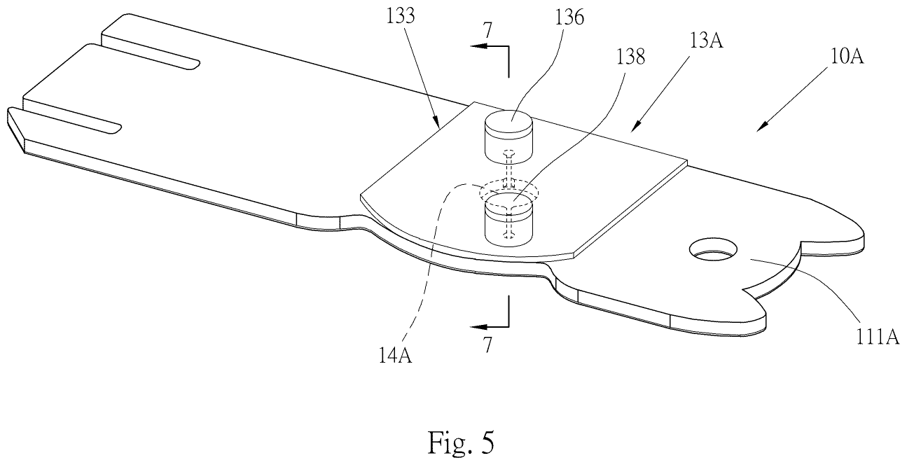

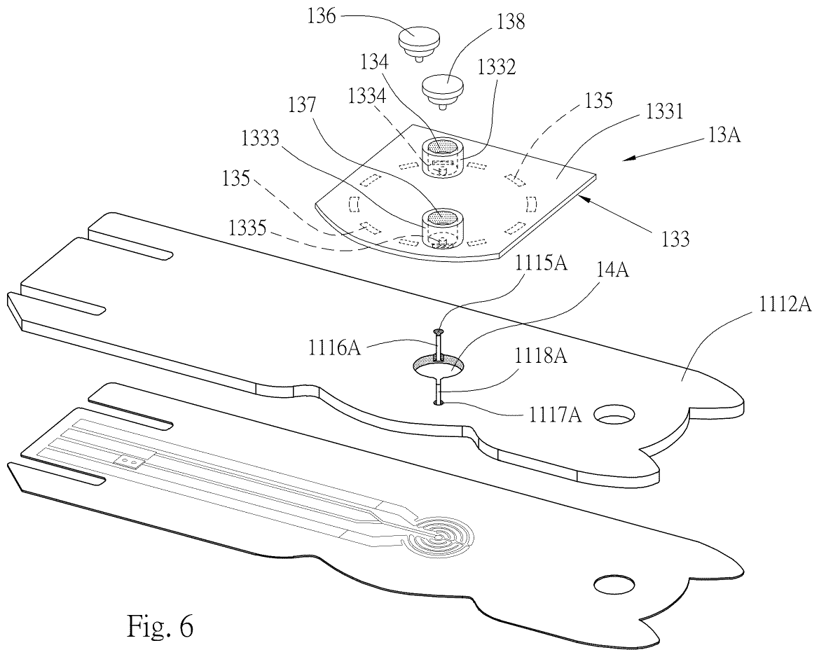

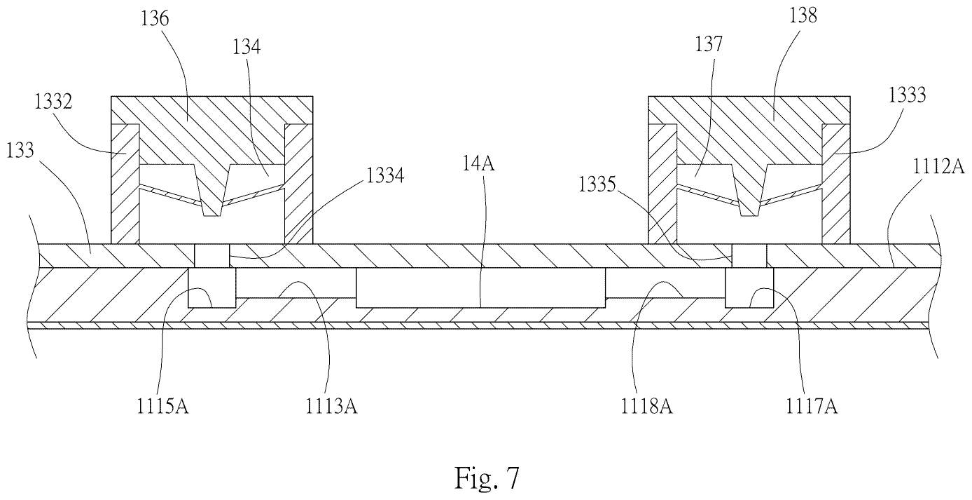

Referring to FIG. 5 to FIG. 7 for the second embodiment of the present invention, the chip 10A disclosed in the second embodiment is different from the chip 10 in the first embodiment mainly in that the packaging portion 13A doubles as a liquid injection base. More specifically, the packaging portion 13A includes a base body 133, a first channel 134, a second adhesive layer 135, a first plug 136, a second channel 137, and a second plug 138. The base body 133 has a substrate 1331, a first tube member 1332, and a second tube member 1333. The first tube member 1332 is provided on one side (hereinafter referred to as the first side) of the substrate 1331, corresponds in position to the injection hole 1115A, and has a tube axis perpendicular to the substrate surface of the substrate 1331, and the other side (hereinafter referred to as the second side) of the substrate 1331 is formed with a first through hole 1334 that is in communication with the internal space of the first tube member 1332, with the internal space of the first tube member 1332 and the first through hole 1334 jointly defining the first channel 134, and the hole diameter of the first through hole 1334 being similar to the hole diameter of the injection hole 1115A. Similarly, the second tube member 1333 is provided on the first side of the substrate 1331, corresponds in position to the outflow hole 1117A, and has a tube axis perpendicular to the substrate surface of the substrate 1331, and the second side of the substrate 1331 is formed with a second through hole 1335 that is in communication with the internal space of the second tube member 1333, with the internal space of the second tube member 1333 and the second through hole 1335 jointly defining the second channel 137, and the hole diameter of the second through hole 1335 being similar to the hole diameter of the outflow hole 1117A.

In addition, the inner wall surface of the first tube member 1332 and the inner wall surface of the second tube member 1333 are each formed with a second hydrophilic surface, and the second hydrophilic surfaces are formed by the same plasma treatment as stated above.

The second adhesive layer 135 is provided on the substrate 1331 by screen printing and is on the same side as the first through hole 1334 and the second through hole 1335. Screen printing is a technique that involves printing with a screen that provides blockage in a negative pattern of the desired pattern; therefore, screen printing can be used to apply a coating in a sophisticated pattern. For example, the second adhesive layer 135 matches the contour of the reaction well 14A and includes an appropriate number of sections that are provided on the substrate 1331 at intervals. In other feasible modes of implementation, the second adhesive layer 135 may instead be provided on the first side 1112A of the plate body 111A and around the reaction well 14A, and with this alternative arrangement, the base body 133 can be adhesively bonded to the plate body 111A just as well. Besides, the technique by which the base body 133 is fixed to the plate body 111A is not limited to adhesive bonding; other fixing methods can produce the same effect.

Once the base body 133 is provided on the first side 1112A of the plate body 111A, the substrate 1331 is laid over the first side 1112A of the plate body 111A and closes the reaction well 14A, the injection channel 1116A, and the outflow channel 1118A at the same time, and the first through hole 1334 and the second through hole 1335 are aligned with and connected to the hole opening of the injection hole 1115A and the hole opening of the outflow hole 1117A respectively. As a result, the first channel 134 and the injection hole 1115A are in communication with each other, and the second channel 137 and the outflow hole 1117A are in communication with each other. In addition, the second adhesive layer 135 is located between the base body 133 and the plate body 111A to bond the base body 133 to the plate body 111A adhesively.

The to-be-detected substance is injected into the reaction well 14A in the following manner. The injection nozzle of the injector with which to inject the to-be-detected substance is inserted into the first tube member 1332 of the base body 133 (the first tube member 1332 being configured for such insertion) until an appropriate degree of tightness of connection between the injection nozzle and the first tube member 1332 is achieved to ensure that the to-be-detected substance will not leak out during the injection process, and that the to-be-detected substance will enter the reaction well 14A sequentially through the first channel 134, the injection hole 1115A, and the injection channel 1116A.

The first plug 136 is removably provided in the end opening of the first tube member 1332 that is far from the plate body 111A, and the first plug 136 is used to cut off communication between the first channel 134 and the outside.

The second plug 138 is removably provided in the end opening of the second tube member 1333 that is far from the plate body 111A, and the second plug 138 is used to cut off communication between the second channel 137 and the outside.

More specifically, the inner wall of each of the first tube member 1332 and the second tube member 1333 is provided with a structure for preventing removal of the corresponding plug. Each of these structures can engage with the corresponding one of the first plug 136 and the second plug 138 to enhance the closure of the injection hole 1115A and of the outflow hole 1117A.

The packaging portion 13A defined in the second embodiment can be used in the packaging technique employed to make the quantitative molecular detection chip in the first embodiment, and in that case, the packaging operation will be performed in a different manner from that stated above.

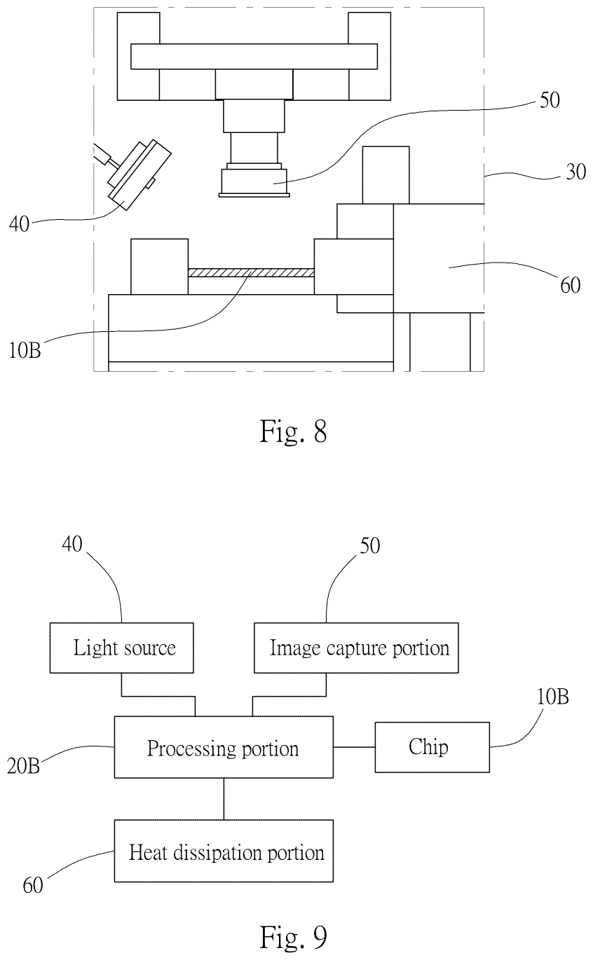

FIG. 8 and FIG. 9 show the quantitative molecular detection system provided by the third embodiment of the present invention. The quantitative molecular detection system includes the chip 10B, the processing portion 20B, a machine frame 30, a light source 40, an image capture portion 50, and a heat dissipation portion 60. The light source 40, the image capture portion 50, the heat dissipation portion 60, and the processing portion 20B can transmit signals to one another, either directly or indirectly, through a wired or wireless medium (e.g., the 4^{th}-generation mobile communication technology (4G), the 5^{th}-generation mobile communication technology (5G), WiFi, Bluetooth, near-field communication (NFC), or radio-frequency identification (RFID)).

The machine frame 30 is the basic structure for supporting the other components. Simply put, the machine frame 30 in this embodiment is assembled essentially from four plates and is where the chip 10B, the light source 40, the image capture portion 50, the heat dissipation portion 60, and the processing portion 20B are placed or installed.

The chip 10B is the quantitative molecular detection chip in the first embodiment or the second embodiment and is used to carry the to-be-detected substance. In this embodiment, the to-be-detected substance includes a biological sample and a polymerase chain reaction (PCR) reagent. The term “biological sample” refers to a sample containing a component of the cells or tissue of a human, an animal, or other organisms, such as blood, plasma, serum, urine, saliva, or feces.

The biological sample is fluorescently tagged with the PCR reagent. Commonly used fluorescent tags include, but are not limited to, FAM, HEX, TET, TAMRA, Cy3, Cy5, Cy5.5, Texas Red, VIC, Yakima Yellow, BHQ-1, BHQ-2, and BHQ-3, with FAM emitting green light, HEX emitting yellow light, and TAMRA emitting red light. In this embodiment, FAM is used for fluorescent tagging, and the fluorescence generated by exciting FAM is in a green-light band that peaks at 520 nm.

The light source 40 may be, but is not limited to, a known light-emitting element for emitting red light, green light, blue light, or any other color light. In this embodiment, the light source 40 is a light-emitting diode (LED) whose model number is VISHAY 78-VLDB1232G-08 and which emits blue light having a wavelength ranging from 458 nm to 472 nm, the blue light preferably peaking at 465 nm. Moreover, when the blue light is projected on the chip 10B, the angle of incidence of the light may be any angle, preferably 45°. The fluorescence generated by exciting the fluorescently tagged to-be-detected substance is in a green-light band peaking at 520 nm and is picked up by the image capture portion 50. What is special about the quantitative molecular detection system is that the curved surface allows the blue light to converge in the reaction well, and that in consequence the light wave signal strength of the fluorescence resulting from the excitation will be increased, thereby improving the prior art problem of producing an unsatisfactory detection result. The blue light can also penetrate the packaging portion 13 and enter the reaction well 14 from the first side 1112 of the plate body 111.

The image capture portion 50 may be, but is not limited to, a charge-coupled device (CCD), a complementary metal-oxide semiconductor (CMOS) device, or another photosensitive device and is used to receive the fluorescence, generate a light wave signal, and transmit the light wave signal to the processing portion 20B. In addition, a filter is provided between the image capture portion 50 and the chip 10B to allow passage of only light of a particular wavelength and to exclude external interfering signals.

The processing portion 20B has at least one microprocessor, at least one central processing unit (CPU), at least one computation device, at least one microcontroller, at least one digital signal processor, at least one graphics processing unit (GPU), another similar device having a computation function or a group of such devices, or a combination of the above and is used to receive the light wave signal and perform a detection analysis, which in this embodiment involves fluorescence detection and in other feasible modes of implementation may involve reflectance measurement or absorbance measurement instead.

The heat dissipation portion 60 is an air-blowing device fixedly provided at a position adjacent to the chip 10B, and the air-blowing device includes a motor and a fan driven by the motor to blow air toward the chip 10B. The heat dissipation portion 60 is controlled by the processing portion 20B in order to cool the chip 10B by air cooling. For example, the fan is activated when the thermal cycle is so controlled that the denaturation temperature (which is a high temperature) is lowered to the annealing temperature (which is a low temperature), and the fan will not be stopped until the temperature of the chip 10B is reduced to the preset annealing temperature.

In terms of practical applications, the quantitative molecular detection system of the present invention can be used for real-time PCR (qPCR) detection. It should be pointed out that, although the foregoing description makes reference to illustrative embodiments for use in specific applications, the claimed invention is not limited to the embodiments described above. By reading the teachings provided herein, a person skilled in the art will be able to know additional modifications, applications, and embodiments that are within the scope of the appended claims.

What is claimed is:

1. An improved structure of a quantitative molecular detection chip, the improved structure comprising:
a base portion;
a reaction well provided in the base portion to receive a to-be-detected substance; and
a light-permeable portion having a curved surface spaced apart from the reaction well, wherein the curved surface has an optical focus located within a well space of the reaction well such that light passing through the curved surface is focused in the reaction well.

2. The improved structure of a quantitative molecular detection chip as claimed in claim 1, wherein the reaction well has a well wall and a well bottom, both formed with a first hydrophilic surface.

3. The improved structure of a quantitative molecular detection chip as claimed in claim 2, wherein the first hydrophilic surface is formed by a plasma treatment.

4. The improved structure of a quantitative molecular detection chip as claimed in claim 1, wherein the curved surface of the light-permeable portion is integrally formed by a peripheral contour surface of the base portion.

5. The improved structure of a quantitative molecular detection chip as claimed in claim 1, wherein the optical focus of the curved surface is at a center of the reaction well.

6. The improved structure of a quantitative molecular detection chip as claimed in claim 1, wherein the reaction well has a circular tube shape, and the optical focus of the curved surface coincides with a center of curvature of the circular tube shape.

7. The improved structure of a quantitative molecular detection chip as claimed in claim 1, wherein the base portion has a first side, the reaction well has a well opening at the first side, and the improved structure further includes:
a packaging portion provided on the first side of the base portion to close the well opening of the reaction well.

8. The improved structure of a quantitative molecular detection chip as claimed in claim 7, wherein the packaging portion comprises:
a film layer laid over the first side of the base portion; and
a first adhesive layer provided between the film layer and the base portion to bond the film layer to the base portion adhesively.

9. The improved structure of a quantitative molecular detection chip as claimed in claim 7, wherein the base portion has a plate body, the plate body has a plate portion, a first side, a second side, an injection hole, an injection channel, an outflow hole, and an outflow channel, the first side and the second side of the plate body are two opposite end faces of the plate portion, the reaction well is provided in the first side of the plate body in a recessed manner, the injection hole is provided in the first side of the plate body in a recessed manner and is spaced apart from the reaction well, the injection channel is provided between the injection hole and the reaction well to enable communication between the injection hole and the reaction well, the outflow hole is far from the injection hole, is provided in the first side of the plate body in a recessed manner, and is spaced apart from the reaction well, and the outflow channel is provided between the outflow hole and the reaction well to enable communication between the outflow hole and the reaction well.

10. The improved structure of a quantitative molecular detection chip as claimed in claim 9, wherein the packaging portion comprises:
a base body; and
a first channel and a second channel corresponding in position to the injection hole and the outflow hole respectively and penetrating the base body, wherein a wall surface of the first channel and a wall surface of the second channel are each formed with a second hydrophilic surface;
wherein when the base body is provided on the first side of the base portion, the first channel and the second channel are aligned with, connected to, and in communication with the injection hole and the outflow hole respectively.

11. The improved structure of a quantitative molecular detection chip as claimed in claim 10, wherein the second hydrophilic surfaces are formed by a plasma treatment.

12. The improved structure of a quantitative molecular detection chip as claimed in claim 10, wherein the packaging portion further comprises a second adhesive layer provided between the base body and the base portion to bond the base body to the base portion adhesively.

13. The improved structure of a quantitative molecular detection chip as claimed in claim 12, wherein the packaging portion further comprises:
a plug removably provided in an opening of the first channel to cut off communication between the first channel and outside, wherein the opening of the first channel is far from the base portion and is formed in an end side of the base body; and
a plug removably provided in an opening of the second channel to cut off communication between the second channel and the outside, wherein the opening of the second channel is far from the base portion and is formed in the end side of the base body.

14. The improved structure of a quantitative molecular detection chip as claimed in claim 7, wherein the base portion has a second side facing away from the first side and further includes:
a temperature regulating portion provided on the second side of the base portion and corresponding in position to the reaction well so as to perform temperature regulation on the to-be-detected substance received in the reaction well; and
a sensing portion electrically connected to the temperature regulating portion and located at a position adjacent to the temperature regulating portion.

15. The improved structure of a quantitative molecular detection chip as claimed in claim 14, wherein the temperature regulating portion and the sensing portion are provided on a main body and are electrically connected, each through a different feedback circuit, to a processing portion in order for the processing portion to receive a signal detected by the sensing portion and to control operation of the temperature regulating portion accordingly.

16. The improved structure of a quantitative molecular detection chip as claimed in claim 14, wherein the temperature regulating portion has a planar heating and cooling loop matching the reaction well in shape and provided on an area of the main body that overlaps with a well bottom of the reaction well.

17. The improved structure of a quantitative molecular detection chip as claimed in claim 16, wherein the sensing portion has a temperature sensing point provided on the main body and corresponding in position to the reaction well, and the planar heating and cooling loop surrounds the temperature sensing point.

18. A quantitative molecular detection system, comprising:
a machine frame;
a chip having the improved structure of any of claim 1 and provided on the machine frame;
a light source provided on the machine frame, wherein the light source generates incident light projected to the reaction well of the chip;
an image capture portion provided on the machine frame to receive fluorescence resulting from excitation with the incident light; and
a heat dissipation portion provided on the machine frame at a position adjacent to the chip to cool the chip.

19. A method of making a quantitative molecular detection chip, the method comprising:
step A: obtaining the base portion of the improved structure of any of claim 1;
Step B: performing a plasma treatment on a well wall of the reaction well provided in the base portion, in order to form a first hydrophilic surface;
Step C: attaching an adhesive patch to the base portion, wherein the adhesive patch carries a temperature regulating portion for heating and cooling the to-be-detected substance received in the reaction well; and
Step D: closing a well opening of the reaction well by a packaging technique.

20. The method of making a quantitative molecular detection chip as claimed in claim 19, wherein the packaging technique comprises: adhesively attaching one side of a film layer to the base portion such that the film layer is adhesively bonded to the base portion and thereby closes the reaction well, wherein the side of the film layer is formed with a first adhesive layer.

21. The method of making a quantitative molecular detection chip as claimed in claim 19, wherein the packaging technique comprises: providing a liquid injection base on the base portion, wherein the liquid injection base has a base body and a channel penetrating the base body, and the channel is in communication with the reaction well when the base body is provided on the base portion; and fitting a plug into the channel to cut off communication between the channel and outside.

22. The method of making a quantitative molecular detection chip as claimed in claim 21, further comprising: performing a plasma treatment on a wall surface of the channel to form a second hydrophilic surface.

23. The method of making a quantitative molecular detection chip as claimed in claim 21, wherein by providing the base body on the base portion, a second adhesive layer is provided between the base body and the base portion to bond the base body to the base portion adhesively.
